(19) United States
(12) Patent Application Publication
(10) Pub. No.: US 2011/0243872 A1
(43) Pub. Date: Oct. 6, 2011

(54) CARBOHYDRATE TREATMENT FOR FRAGRANCED ARTICLES

(75) Inventors: Richard Skrobanski, Middletown, NY (US); Dina Piersawl, Northbrook, IL (US); Stephen E. Schuh, Northbrook, IL (US); Norman J. Appel, Middletown, NY (US)

(73) Assignee: BELL FLAVORS AND FRAGRANCES, INC., Northbrook, IL (US)

(21) Appl. No.: 13/139,346

(22) PCT Filed: Dec. 18, 2009
(86) PCT No.: PCT/US09/68619

§ 371 Date: Jun. 13, 2011

Related U.S. Application Data
(60) Provisional application No. 61/141,598, filed on Dec. 30, 2008.

(51) Int. Cl. A61K 8/73; A61L 101/50; A61L 101/48; A61K 8/81; A61Q 13/00; A61Q 5/00; A61Q 19/00; B05D 3/02; B05D 3/10; A01K 29/00

(52) U.S. Cl. 424/70.13; 514/778; 424/76.6; 427/384; 427/385.5; 119/165

(57) ABSTRACT

A carbohydrate treatment composition composing modified starches, preservatives, an incorporation factor and water The treatment composition may also preferably comprise a fragrance The composition may be used to coat a litter material substrate and also may be used in a fragrance delivery composition for personal care products A method of coating a litter material using a carbohydrate material is also presented.

### FIELD OF THE INVENTION

The invention is a fragranced animal litter and a process for preparing fragranced animal litter. The invention is also a carbohydrate treatment for fragranced hair or skin care products.

### DESCRIPTION OF RELATED ART

Prior art teaches the formation of animal litter material which can include binders. Prior art also teaches the coating of the litter material with a variety of materials which may include carbohydrate that are applied with or without fragrance. Prior art also teaches the inclusion of fragrances in the mixture step and inclusion of fragrances in the coating step. Uncoated/untreated cat litter absorbs but does not retain the fragrance long enough to be effective against malodorants in the litter box. The prior art does not teach separate steps of forming the litter substrate, application and drying a carbohydrate coating and application of fragrance to the coated, dried, material, where the purpose of the coating is to enhance the effectiveness of the fragrance.

Prior art also teaches that benefit agents can be deposited on hair, and skin to deliver beneficial effects possibly utilizing the invention. The same prior art may require residence time on hair or skin to deliver benefit agents.

### SUMMARY OF INVENTION

The present invention is related to a carbohydrate treatment composition comprising modified starches, preservatives, an incorporation factor, and water.

The present invention is also related to a method of coating a litter material substrate comprising obtaining a litter material substrate; preparing a carbohydrate treatment composition comprising modified starches, preservatives, an incorporation factor, and water; coating the litter material substrate with the composition; and drying the coating.

The present invention is further related to a fragrance delivery composition for hair or skin comprising a carbohydrate treatment composition comprising modified starches, preservatives, an incorporation factor, and water; a personal care matrix; and a fragrance.

Furthermore, the method coating a litter material substrate such as a clay or similar animal litter, such as cat litter, preferably uses a carbohydrate mixture, comprising 5% to 30% modified starch, 5% to 20% liquid acrylic polymer, 3% to 20% glycol ether and/or glycerin incorporation factors. The coating is applied to the litter material substrate and dried. After drying the coating, the step of applying liquid fragrance preparations to the treated substrate is accomplished. Without being bound by theory, it is believed that taking clay litter, coating and then applying fragrance improves fragrance performance by allowing the fragrance to be retained by the coating on the animal litter long enough to act on the malodorants in the litter box. The preferred fragrance contains 5% to 30% PPG 34 polymer functioning as fragrance adhesion factor and 25% to 50% ethoxylated sorbitan monolaurate fragrance deposition aid further enhancing fragrance retention.

### BRIEF DESCRIPTION OF THE DRAWINGS

In the drawings,

FIG. 1 is a graph of the effect of Aromacote™ on the deposition of fragrance on hair;

FIG. 2 is a graph showing the effect of Aromacote™ concentration on fragrance deposition; and

FIG. 3 is a graph illustrating the difference in fragrance deposition with and without Aromacote™.

### DETAILED DESCRIPTION

A carbohydrate treatment composition can be used for coating animal litter. Several examples of the method of coating the litter as well as of the composition itself are provided below. These examples are not meant to be limiting but are merely representative examples.

A pelletizer can be used as a coating device. Other devices can be used as a coating device for applying the carbohydrate treatment composition.

For instance, a DP-14 Laboratory pelletizer available from Mars Mineral Division of Woodward, Inc. is an inclined pan, variable angle, variable-speed pelletizing device. This pelletizer can also be configured and used as a coating device.

The pelletizer has a plow support arm to which are attached two (2) sets of scraper plows. The function of the plows is to set and maintain a thin coating of material on the back of the pan.

To configure the pelletizer as a coating device, the pelletizer scraper plow support arm is removed, by-passing the pelletizer spraying system attached to it. The coating material can be applied as a liquid or pumped through a nozzle and sprayed. Spray is the preferred process to apply coating material on the clay litter.

### Coating Example 1

Non-limiting Example 1 uses Preparation 1 which is heated until it becomes liquid and applied using the pelletizer. The composition of Preparation 1 is:

| Material | Amount (by weight) |
|---|---|
| Modified Starches | 30% |
| Preservatives | 0.6% |
| Glycol Ether | 19.4% |
| Water | 50% |

Glycol Ether is used as an incorporation factor. Preparation 1 thickens upon standing. To use, the material is heated until liquefied and then ladled onto the clay litter in the pelletizer as configured for coating. Coating is done at ambient conditions.

The pelletizer is set with the pan 14 inches diameter and 9 inches deep. The pan rotates at a speed of 15 rpm calculated (30% setting) where 100% setting is equivalent to 50 rpm. The pan angle is set at 30°. The scraper plow support arm is removed.

The procedure in Example 1 is:
1. Weigh 1500 grams of clay cat litter and charge the pan.
2. Turn on the pan and set spin speed at 30% setting (15 rpm)
3. Ladle 300 g of warmed coating preparation 1 onto the clay litter.
4. Let coated cat litter tumble until dry.

### Coating Example 2

Non-limiting Example 2 uses preparation 2 in an attempt to thin Preparation 1 for spray coating clay litter. The composition of Preparation 2 is:

| Material | Amount (by weight) |
|---|---|
| Modified Starches | 20% |
| Acrylic Polymer | 10% |
| Preservatives | 0.6% |
| Glycol Ether | 19.4% |
| Water | 50% |

The Acrylic Polymer is 19% to 21% solids in water as received. Glycol Ether is used as an incorporation factor.

Preparation 2 thickens upon prolonged standing. However, for use immediately after material preparation, no heating is required. When used after prolonged standing, Preparation 2 can be heated until liquefied and then sprayed onto the clay litter in the pelletizer configured for coating, also using the pelletizer spray system attached to scraper plow support arm.

The pelletizer is set up with the pan 14 inches in diameter and 9 inches deep. The pan speed is 15 rpm calculated (30% setting), where 100% setting is equivalent to 50 rpm. The pan angle is 30°. The pelletizer is fitted with a spray nozzle, it being found that a Unijet® Spray Nozzle from Spraying Systems® Co., ¼ inch NPT, TPU flat spray, 400050 (40 spray pattern), tip size 05 (0.018 inch orifice size) is suitable. As described above, the scraper plow support arm and attached pelletizer Spray System was used.

Preparation 2 was pumped through spray nozzle using a Simon Model Varistaltic Pump from Manostat Corp. fitted with ⅛ inch Tygon™ tubing in the pump head. 5/16 inch Tygon™ tubing was used outside the pump head to draw coating material from a container and connect to nozzle assembly via 5/16 inch connector. The pump flow speed is set at Setting 2, resulting in a coarse spray.

The procedure in Example 2 is:
1. Weigh 1500 grams of clay cat litter and charge the pan.
2. Turn on the pan and set spin speed at 30% setting (15 rpm)
3. Pump 300 g of coating preparation 2 onto the clay litter.
4. Let coated clay tumble until dry.

### Coating Example 3

### Preferred Embodiment for Litter

Non-limiting Example 3 illustrates the use of Preparation 3, which results from thinning Preparation 2 for spray coating clay litter. The composition of Preparation 3 is:

| Material | Amount (by weight) |
|---|---|
| Modified Starches | 5% |
| Acrylic Polymer | 10% |
| Preservatives | 0.6% |
| Glycerin | 3% |
| Water | 81.4% |

The Acrylic Polymer is 19% to 21% solids in water as received. Glycerin is used as an incorporation factor. Optimum use for the coatings are between 10% and 15% to the litter.

This mixture does not thicken on long-term standing and is suitable for spray coating onto the clay litter in a model DP-4 laboratory size pelletizer configured for coating.

### Coating Example 3

### Procedure at Ambient Conditions

The pelletizer is set up with the pan 14 inches in diameter and 9 inches deep. The pan speed is 15 rpm calculated (30% setting), where 100% setting is equivalent to 50 rpm. The pan angle is 30°. The scraper plow support arm removed. In Example 3, the spray nozzle used was a Lurmark® Spray Nozzle from U.S. Plastics Corp. ¼ inch NPT, size 02 (0.035 inch orifice) with a 40 degree spray pattern PVDF Nozzle.

In Example 3, Preparation 3 was pumped through the spray nozzle described above using a Simon Model Varistaltic Pump from Manostat Corp. fitted with 5/16 inch Tygon™ tubing in the pump head. 5/16 inch Tygon™ tubing was used outside the pump head to draw coating material from a container and connect to nozzle assembly via 5/16 inch connector.

At pump flow-speed Setting 2 a coarse spray resulted with greater spray efficiency than that in Example 2.

The procedure in example 3 is:
1. Weigh 1500 grams of clay cat litter and charge the pan.
2. Turn on the pan and set spin speed at 30% setting (15 rpm)
3. Spray 225 g of coating preparation 3 onto the clay litter.
4. Let coated clay tumble until dry.

### Additional Coating Materials

Fatty acid-fatty alcohol and waxy mixtures by engineering to spray and maintain materials at elevated temperature (at least 75° C.).

Cat litter can conceptually be coated with other hydrocolloid materials and synthetic polymers capable of forming films such as gums, cellulosic materials, and other carbohydrates, acrylates/acrylamide, polyethylene, polypropylene, polyurethane, and copolymers and derivatives. These materials may be added to the fragrance as necessary.

When the steps described herein are performed, the resulting coated litter improves liquid fragrance retention on the litter when compared to uncoated cat litter. Liquid fragrance intensity is increased on the coated litter, preferably cat litter, when measured and compared to uncoated cat litter by organoleptic means. There is less malodor perceived in the coated and fragranced litter when compared organoleptically to uncoated and fragranced litter. In this way coated litter improves fragrance retention when compared to uncoated litter.

Coated litter enables the preparation of clay particle fragrance delivery systems not possible with uncoated clay. A manufacturer's cat litter or other similar material can be used as a value added ingredient allowing for high fragrance loading (at least 10%) and be decorative at the same time. This is not possible with uncoated cat litter.

The coating and fragrance application procedures can be adapted to current litter manufacturing processes with minimal engineering and process changes. Resulting coated and fragranced litter particles are essentially dust-free. This effect is also beneficial in manufacturing as litter manufacturing plants are very dusty. The addition of a highly fragranced and coated cat litter piece during cat litter manufacturing would not be irritating because of the non-dusty nature of the particles.

### Fragrance Materials and Fragrance Selection

A fragrance can preferably be added to the carbohydrate treatment composition. Fragrances are mixtures of synthetic and natural substances known to those skilled in the art. Suitable fragrance substances can be found in published works such as but not limited to: Arctander, Steffen, Perfume and Flavor Chemicals, Vol. 1 and 2, Steffen Arctander, Publisher, 1982. Legal reprint printed in Denmark by Det Hoffensbergske Etablissement, Copenhagen V., Denmark, and Perfume and Flavor Materials of Natural Origin, ibid., 1982. Legal reprint printed in Denmark by Det Hoffensbergske Etablissement, Copenhagen V., Denmark.

Fragrance materials are available from a variety of industry sources including manufacturers and distributors of the same and include materials such as aliphatic, acyclic and cyclic terpene, cycloaliphatic, aromatic, and heterocyclic hydrocarbons, alcohols, aldehydes, acetals, ketones, acids, esters and phenol and phenol derivatives, natural extractives and essential oils, and other materials known to those skilled in the art.

Suitable proprietary fragrance compositions are produced by skilled practitioners of the perfumery art that are specifically tailored to gain maximum performance in the desired application. Broad knowledge of fragrance material olfactive and physical properties such as but not limited to odor quality, vapor pressure, cLogP, stability in and reactivity and affinity with the desired substrate greatly influences the fragrance composition. The result is a pleasant fragrance accepted by the consumer with the performance criteria desired of the consumer product. Therefore, it is known to those skilled in the art, that a perfume for a household product such as cat litter will be different from a perfume for a personal care product such as shampoo.

Fragrance Preparation—Cat Litter Example

| Material | Amount (by weight) |
|---|---|
| Fragrance | 70% |
| PPG 34 Polymer | 5% |
| Ethoxylated Sorbitan | 25% |
| Monolaurate | |

In the non-limiting fragrance preparation, the fragrance can be, but is not limited to, one or a combination of herbal types, citrus types or fresh types, etc. The PPG 34 Polymer is used as a fragrance adhesion factor. The Ethoxylated Sorbitan Monolaurate is used as a fragrance deposition aid.

The fragrance preparation of paragraph [00035] is used with each of the Examples 1, 2 and 3 and their respective coating preparations 1, 2, and 3. Preparations 1, 2 and 3 are shown together in the following table:

| Material | Example 1 | Example 2 | Example 3 |
|---|---|---|---|
| Modified Starches | 30% | 20% | 5% |
| Acrylic Polymer | | 10% | 10% |
| Preservatives | 0.60% | 0.60% | 0.60% |
| Glycol Ether | 19.40% | 19.40% | |
| Glycerin | | | 3% |
| Water | 50% | 50% | 81.40% |

The procedure for applying the fragrance (in the laboratory), after completion of the steps described above in Examples 1, 2 and 3 is:
1. Weigh ½ of the desired amount of coated clay litter prepared pursuant to Examples 1, 2 or 3 into a 1 quart jar.
2. Add ½ of the desired amount of fragrance preparation and mix with spatula until well mixed
3. Add the remaining amount of coated litter and remaining amount of fragrance preparation on top of step 2 immediately above and mix well.
4. Cover and roll the jar to tumble contents on a Fasco Industries, Model 71637283 Type U63 Tumbler until contents are dry.

Fragrance preparation usages range from 0.5% to 2.5% for the described above procedure. Preferably, the usage range is around 1.0% and 1.1%.

TABLE 1
Organoleptic Scores for Fragranced Animal Litter: Uncoated
and Coated at 13% [00024] According to the Invention with
Fragrance Preparation [00035] at 1.06%.

| | | Animal |
|---|---|---|
| | Fragrance | Malodor |
| Sample | Intensity | Intensity |
| Herbal Type Fragrance Uncoated | 35.6 | 11 |
| Herbal Type Fragrance Coated | 41.3 | 13.5 |
| Fresh Type Fragrance Uncoated | 29.3 | 22.1 |
| Fresh Type Fragrance Coated | 43 | 10.7 |
| Fresh Type Fragrance II Uncoated | 33.2 | 17 |
| Fresh Type Fragrance II Coated | 39.4 | 15.3 |
| Clean Type Fragrance Uncoated | 25.6 | 18.1 |
| Clean Type Fragrance Coated | 37.8 | 9.6 |

Litter preparation for organoleptic testing: The litters are placed in a tumbler to which fragrance preparation is sprayed. The fragranced litter is allowed to set for 1 week. Then the litter is treated with liquid and solid animal odorants and allowed to set uncovered for 1 week. Following the last set, the odorants are removed from the litter. The litter is spread on a tray to remove clumps. The litter is then split to a representative 4 ounce quantity into a 4 ounce specimen cup with lid. The samples are presented to a fragrance panel to judge fragrance intensity and malodorant intensity. High fragrance intensity and low malodorant intensity scores are desired.

Results: Fresh Type fragrance II and Clean Type are more efficient covering malodor with a higher fragrance score than the herbal fragrance and lower malodor score. To those skilled in the art, the herbal fragrance could be optimized to yield a lower malodor score.

In an industrial setting, the fragrance material may be sprayed with the same equipment as described to spray coating preparation 3 (above), taking care to eliminate fragrance contamination when applying different fragrances successively.

Highly Dosed Litter Particles Diluted with Uncoated Litter Particles

In another embodiment, the coated litter material substrate was used as a fragrance delivery system, where the fragrance preparation is applied as described at dosages of 5% to 20%. These highly dosed clay litter particles can then be diluted to the desired fragrance levels with the bulk of uncoated clay, thereby reducing or eliminating irritating dust experienced with applying powdered fragrances in an industrial cat litter manufacturing setting.

### Preparation of Colored Clay Pieces

In another embodiment, colored animal litter can be produced pursuant to the following procedure.

Clay litter is coated with coating preparation 3 according to Example 3 and let dry, and subsequently:
1. This coated clay litter is then coated at 10% wt. with polytetrafluoroethylene (PTFE), 60% aqueous dispersion with adhesion additive as received from Laurel Products, Elverson, Pa. White coating is visible on the clay litter particles.
2. The PTFE coated clay litter is, in turn, let to dry.
3. A green dye dilution is formed in which 1% wt. green liquid dye (as purchased) is blended with 99% wt. benzyl benzoate and mixed well until uniform. Solvents other than benzyl benzoate may also be used so long as they are compatible at the same time with fragrance and green liquid dye.
4. The green dye dilution (4 above) is blended at 50/50 by wt. with fragrance preparation and mixed well until uniform.
5. To 82.5 g coated and dried clay litter, steps 2 and 3 (above), 17.5 g of colored fragrance, step 5 (above) is applied in a jar as described in the fragrance preparation step described above.
6. A green colored clay litter particle results that can be blended to bulk uncoated clay litter to add decorative pieces to the bulk uncoated cat litter.

It is understood by those skilled in the art, embodiments may need adjustment to meet the requirements for consumer products not limited by government regulations, interactions with matrices and fragrances, consumer fragrance preferences, etc. The wording “Aromacote™ will be used herein and is the descriptor for an embodiment of a carbohydrate treatment thereof.

This invention is also related to a carbohydrate treatment composition for delivering fragrance to hair or skin. A composition that comprises such a carbohydrate treatment composition is referred to as a fragrance delivery composition and comprises a carbohydrate treatment composition, a personal care matrix, and a fragrance.

Personal Care Example: Shampoo

Surprisingly, by simple mixing Aromacote™ to a fragranced shampoo matrix, a fragrance enhancement was found. Similar results would be expected with changes to the shampoo matrix by those skilled in the art.

SHAMPOO MATRIX

| Ingredients | Amount (% w/w) |
|---|---|
| Deionized Water | QS to 100.00 |
| Sodium Lauryl Sulfate | 30.00 |
| Cocamidopropyl Betaine | 5.00 |
| Cocamide DEA | 3.00 |
| Pearlizing Blend | 2.00 |
| DMDM Hydantoin | 0.40 |
| Fragrance | As required by test |
| Aromacote ™ | As required by test |
| Citric Acid | QS to pH: 6.5-6.8 |

Fragrance Materials Tested

Fragrance compounds were selected for their affinity for hair and mixed in equal proportions. Shampoos were formulated according to [00044] with 1.0% w/w fragrance materials and varying amounts of Aromacote™ as follows:

FRAGRANCE MATERIAL MIXTURE

| Material | Amount (% w/w) |
|---|---|
| Styrene | 20.00 |
| Camphor | 20.00 |
| Verdox ™ (1) | 20.00 |
| Lilial ™ (2) | 20.00 |
| Benzyl Benzoate | 20.00 |
| Total | 100.00 |

(1) Trademark of International Flavors and Fragrances, Inc. Chemical name: 2-tert-Butylcyclohexylacetate
(2) Trademark of Givaudan SA Chemical name: 3-(4-tert-Butylphenyl)-2-methylpropionaldehyde

FRAGRANCED SHAMPOO MATRIX [00045] WITH VARYING
AMOUNTS OF AROMACOTE ™ AND 1% W/W
FRAGRANCE MIXTURE [00046]:

| Test | Shampoo Matrix | Fragrance Mixture | Aromacote ™ |
|---|---|---|---|
| Number | [00044] (%) w/w) | [00045] (% w/w) | (% w/w) |
| 1 | 99.00 | 1.00 | 0.00 |
| 2 | 98.50 | 1.00 | 0.50 |
| 3 | 96.50 | 1.00 | 2.50 |
| 4 | 94.00 | 1.00 | 5.00 |
| 5 | 89.00 | 1.00 | 10.00 |
| 6 | 84.00 | 1.00 | 15.00 |

Procedure
Hair tresses were obtained and were shampooed with each shampoo matrix, rinsed and dried under ambient conditions.
Fragrance retention on the hair tresses was analyzed by Twister extraction Headspace analysis as follows:
Approximately 0.2 g of hair was weighed into a headspace vial.
Water was sprayed onto the hair followed by the addition of 0.75 ug of Carvyl Acetate as an internal standard (approximately 5 ppm of the hair).
A Twister extraction bar was suspended over the sample.
The sample was heated to 80° C. for 1 hour, followed by chromatographic analysis.
Each sample was prepared and analyzed four times.

Results

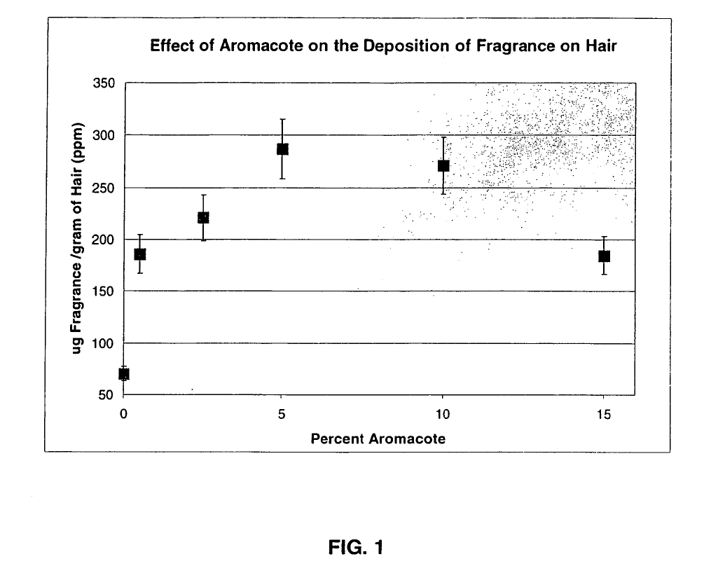

FIG. 1 is a graph illustrating the results of the effect of Aromacote™ on the deposition of fragrance on hair. The results are reported as averages. Relative standard deviation is about 10%.

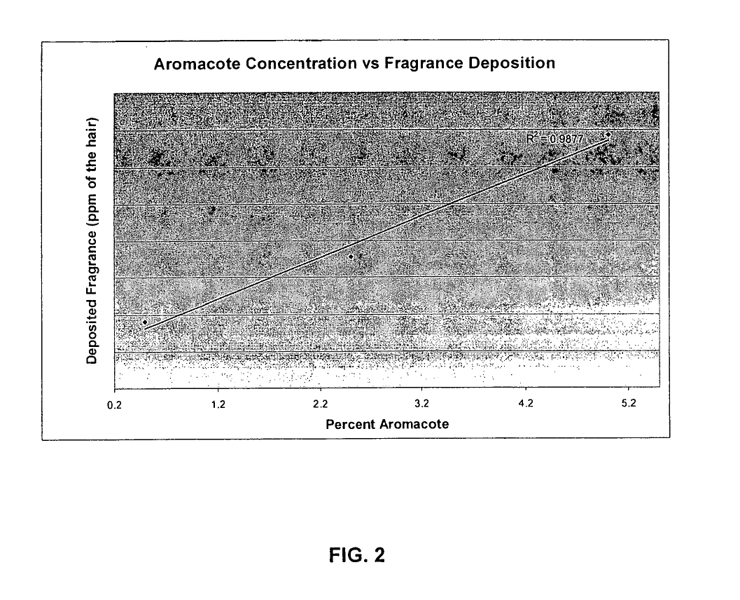

FIG. 1: Shows the Effect of 0%-to-15% Aromacote™ on the Deposition of 1% Fragrance Mixture [00045] via shampoo matrix [00044].
Fragrance deposition is linear between 0.5% and 5% Aromacote™ (see FIG. 2).
Deposition plateaus at 5% Aromacote™ via shampoo matrix [00044]
About four times more fragrance is deposited on the hair at 5% Aromacote™ in shampoo matrix [00044] compared to control shampoo matrix [00044] without Aromacote™
There is no difference in fragrance deposition on hair between 5% and 10% Aromacote™ in shampoo matrix.
At 15% Aromacote™ in shampoo matrix, fragrance deposition on hair is reduced.
Above 5% Aromacote™ in shampoo matrix, the matrix ceases to be a shampoo.
   FIG. 2 is also a graph which illustrates the concentration of Aromacote™ and how it changes the deposition of the fragrance. FIG. 2 shows linearity of Aromacote™ hair treatments via shampoo between 0.5% and 5.0% in shampoo

Fragrance Types Tested

Based on the results in FIG. 1, a herbal green fragrance, a fruity floral musk fragrance and a cucumber green tea fragrance was tested in the same shampoo matrix at 1% and 5% Aromacote™. The procedures for hair tress treatment and analysis are described in [00046].

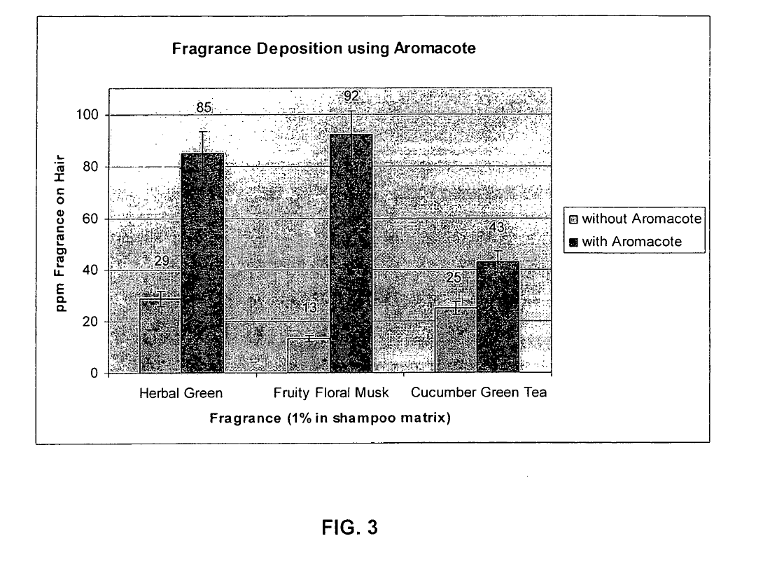

FIG. 3 is a graph that illustrates the difference between fragrance deposition when using Aromacote versus not using Aromacote™. FIG. 3 shows the deposition of fragrance types at 1% in shampoo matrix [00044] containing 5% Aromacote™

Aromacote™ Results Summary: Fragrance deposition on hair is improved with the addition of Aromacote™ in shampoo matrix. Not being bound by any theory, when Aromacote™ is formulated into a consumer product, the product containing Aromacote™ and fragrance is activated when the consumer uses the desired product and then is washed away after activation leaving behind a desired fragrance. The same effect is reduced when Aromacote™ is not present in the same product. It is not required that Aromacote™ is left on the skin or hair to retain the desired fragrance.

While several embodiments have been shown and described with respect to the present invention, it should be understood that the present invention is not limited to these embodiments, but rather is susceptible to numerous changes and modifications as known to those skilled in the art. Therefore, we do not wish to be limited to the detail shown and described herein, and intend to cover all such changes and modifications as are encompassed by the scope of such claims as may be appended.

We claim:

1. A carbohydrate treatment composition comprising modified starches, preservatives, an incorporation factor, and water.

2. The composition as in claim 1 further comprising an acrylic polymer.

3. The composition as in claim 2 wherein the acrylic polymer is about 5% to about 20% by weight of the composition.

4. The composition of claim 2 wherein the acrylic polymer is from about 19% to about 21% solids in water when received.

5. The composition of claim 2 wherein the acrylic polymer is a liquid.

6. The composition as in claim 1 or 2 wherein the modified starches are from about 5% to about 30% by weight of the composition.

7. The composition of claim 1 or 2 wherein the incorporation factor is selected from the group consisting of glycol ether and glycerin.

8. The composition of claim 1 or 2 wherein the incorporation factor is from about 3% to about 20% by weight of the composition.

9. The composition of claim 2 further comprising a fragrance preparation.

10. The composition of claim 9 wherein the fragrance preparation comprises a fragrance material, a fragrance adhesion factor, and a fragrance deposition aid.

11. The composition of claim 9 wherein the fragrance material is selected from the group consisting of aliphatic terpene, acyclic terpene, cyclic terpene, cycloaliphatic hydrocarbons, aromatic hydrocarbons, heterocyclic hydrocarbons, alcohols; aldehydes; acetals, ketones, acids, esters, phenol derivatives, natural extractives and essential oils, and a combination of the aforementioned.

12. The composition of claim 10 wherein the fragrance material is selected from the group consisting of herbal types, citrus types, and fresh types.

13. The composition of claim 10 wherein the fragrance adhesion factor is PPG 34 Polymer.

14. The composition of claim 10 wherein the fragrance deposition aid is ethoxylated sorbitan monolaurate.

15. The composition of claim 9 wherein the fragrance preparation is in a range of from about 0.5% to about 20%.

16. The composition of claim 15 wherein the fragrance preparation is in a range of from about 0.5% to about 2.5%.

17. The composition of claim 15 wherein the fragrance preparation is in a range of from about 1.0% to about 1.1%.

18. The composition of claim 10 wherein the fragrance adhesion factor is in an amount of between about 5% to about 30%.

19. The composition of claim 10 wherein the fragrance deposition aid is in an amount of between about 25% and 0%.

20. A method of coating a litter material substrate comprising:
obtaining a litter material substrate;
preparing a carbohydrate treatment composition comprising modified starches, preservatives, an incorporation factor, and water;
coating the litter material substrate with the composition; and
drying the coating.

21. The method of claim 20 further comprising the step of adding a liquid fragrance preparation after the drying step.

22. The method of claim 20 further comprising the step of preparing a pan with the litter material substrate.

23. The method as in claim 20 wherein the litter material substrate is a clay animal litter.

24. The method as in claim 20 wherein the litter material substrate is cat litter.

25. The method as in claim 20 wherein the composition comprises about 5% to about 30% modified starch, about 5% to about 20% liquid acrylic polymer, and about 3% to about 20% incorporation factors.

26. The method as in claim 20 wherein the incorporation factors are selected from the group consisting of glycol ether and glycerin.

27. The method of claim 21 wherein the liquid fragrance preparation comprises a fragrance material, a fragrance adhesion factor, and a fragrance deposition aid.

28. The method of claim 27 wherein the fragrance adhesion factor of the liquid fragrance is PPG 34 polymer in an amount of about 5% to about 30%.

29. The method of claim 27 wherein the fragrance deposition aid is ethoylated sorbitan monolaurate in an amount of about 25% to 50%.

30. The method of claim 20 wherein the coating step is applied from the group consisting of spraying, pouring, or pumping.

31. The method of claim 20 wherein the coated substrate is dried by tumble drying.

32. The method of claim 23 further comprising the step of blending the coated animal litter with bulk litter.

33. A fragrance delivery composition for hair or skin comprising:
a carbohydrate treatment composition comprising modified starches, preservatives, an incorporation factor, and water;
a personal care matrix; and
a fragrance.

34. The fragrance delivery composition wherein the carbohydrate treatment composition is in an amount of from about 0.5% to about 15%.
